(19) United States
(12) Patent Application Publication
(10) Pub. No.: US 2008/0091186 A1
(43) Pub. Date: Apr. 17, 2008

(54) Electro-surgical device RF energy needle electrical shorting plate

(75) Inventor: Jeffrey Wayne Allison, Etters, PA (US)

Correspondence Address: TYCO TECHNOLOGY RESOURCES, 4550 NEW LINDEN HILL ROAD, SUITE 140, WILMINGTON, DE, 19808-2952, US

(73) Assignee: TYCO ELECTRONICS CORPORATION, Middletown, PA (US)

(21) Appl. No.: 11/549,327

(22) Filed: Oct. 13, 2006

(51) Int. Cl. A61B 18/14

(52) U.S. Cl. 606/32; 606/41

(57) ABSTRACT

A shorting plate to accommodate multiple needles and provide a secure electrical connection. The plate provides an electrical charge to all needles of an electro surgical device with reduced time and costs in manufacturing. Protrusions formed in the apertures receive the surgical needles and make a secure electrical connection without a permanent connection.

### BACKGROUND OF THE INVENTION

The present invention is directed to an electro surgical device, and more specifically an electrical shorting plate for surgical needles.

To effectively remove or treat a tumor with an electro surgical needle device, a surgical needle is given an electrical current. This electrically charged needle is placed in or near the tumor where the heat generated from the current in the needle burns the tumor until it disintegrates or is cut away from healthy tissue. Often in some surgical devices, several needles are used within a single device to treat larger areas than a single needle can treat at once.

In addition to treating tumors, the electro surgical devices may be used for other applications as well. These applications may include, but are not limited to, other abnormal growths, opening, clogged blood vessels in cardiovascular surgery and debriders for use on polyps in ear, nose and throat surgery. Still other applications include instruments include, for example, surgical laser systems, cryosurgical systems, and electrosurgical systems.

One method and system for using multiple surgical needles in one device involved providing an electrical current to one needle and electrically shoring the other needles to the charged needle. The shorting is achieved by wrapping conductive material, such as wire, around the multiple needles, including the charged needle, which results in all the needles being electrically charged.

Another method and system for using multiple needles uses the same wire wrapping technique and further solders the wire to the needles for a more secure connection. The wire wrapping and soldering connection are all contained within a handle or protective layer to ensure that only the ends of the needles are exposed. These methods and systems are time consuming and expensive to manufacture.

Therefore, what is needed is a system for shorting together multiple electrical surgical needles in a more time effective and cost effective manner while still providing a secure electrical connection between the multiple surgical needles.

### SUMMARY OF THE INVENTION

One embodiment of the present invention includes an electrical shorting plate having an electrically conductive body and a plurality of apertures formed in the electrical body having at least one protrusion disposed at an angle from the body. The body conducts an electrical charge to the plurality of protrusions, the apertures are configured to receive an electrically conductive element, the at least one protrusion resiliently receives the electrically conductive element and maintains a secure electrical connection with the electrically conductive element.

Another embodiment of the present invention includes an electrical surgical device having an electrical power source and a plurality of electrically conductive elements where one of the electrically conductive elements is connected to the electrical power source and configured to receive an electrical charge. The embodiment also includes an electrical shorting plate having an electrically conductive body and a plurality of apertures formed in the electrical body, each aperture having at least one protrusion disposed at an angle from the body. The body conducts an electrical charge to the at least one protrusion, the apertures are configured to receive an electrically conductive element, the at least one protrusion resiliently receives the electrically conductive element and maintains a secure electrical connection with the electrically conductive element.

One advantage of the present invention is improved manufacturing and reduced assembly time.

Another advantage of the present invention is reduced costs for manufacturing.

Yet another advantage of the present invention is the solid and secure electrical connection between the multiple needles.

Other features and advantages of the present invention will be apparent from the following more detailed description of the preferred embodiment, taken in conjunction with the accompanying drawings which illustrate, by way of example, the principles of the invention.

### BRIEF DESCRIPTION OF THE DRAWINGS

FIG. 1 illustrates the existing technology related to the present invention.

FIG. 2 illustrates the present invention as used in a surgical needle device.

FIG. 3A illustrates one embodiment of a conductive plate of the present invention.

FIGS. 3B and 3C illustrate opposite views of the conductive plate of FIG. 3A receiving a conductive element of the present invention.

FIG. 4 illustrates a plan view of another embodiment of the conductive plate of the present invention.

FIG. 5 illustrates a side view of the conductive plate of FIG. 4 of the present invention.

FIG. 6 illustrates a perspective view of the present invention.

Wherever possible, the same reference numbers will be used throughout the drawings to refer to the same or like parts.

### DETAILED DESCRIPTION OF THE INVENTION

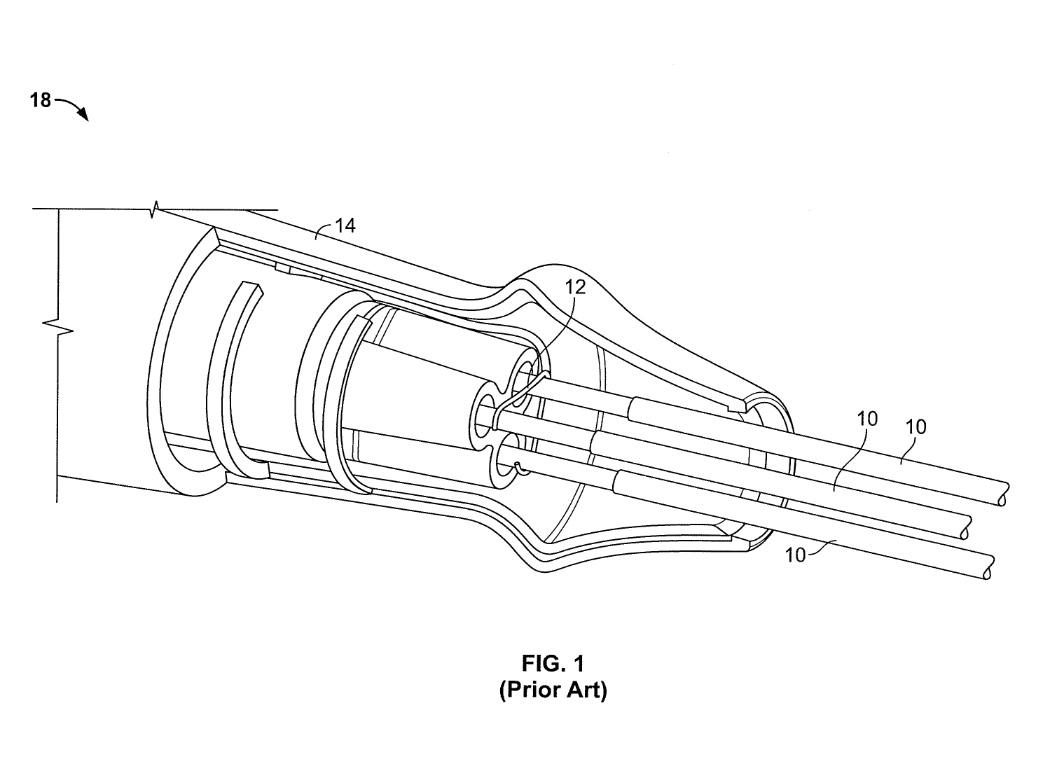

An application similar to the present invention in prior art is illustrated in FIG. 1 where an electro surgical device 18 houses multiple RF energy needles 10. The RF energy needles 10 are electrically connected with a wire 12 wrapped around and connected to all three needles 10. One of the needles 10 is electrically charged by an electrical connection provided from a cord 16 that extends into the handle 14. The wire 12 makes contact with the electrically charged needle 10 and conducts the charge to the other needles 10 when the connection is made between the wire 12 and the needles 10. This mutual electrical connection causes the plurality of needles 10 and not just a single needle to be charged and capable of treating tumors or performing other medical tasks. Typically, a solder connection is made where the wires 12 connect to the needles 10 to ensure a more secure electrical connection than wrapping.

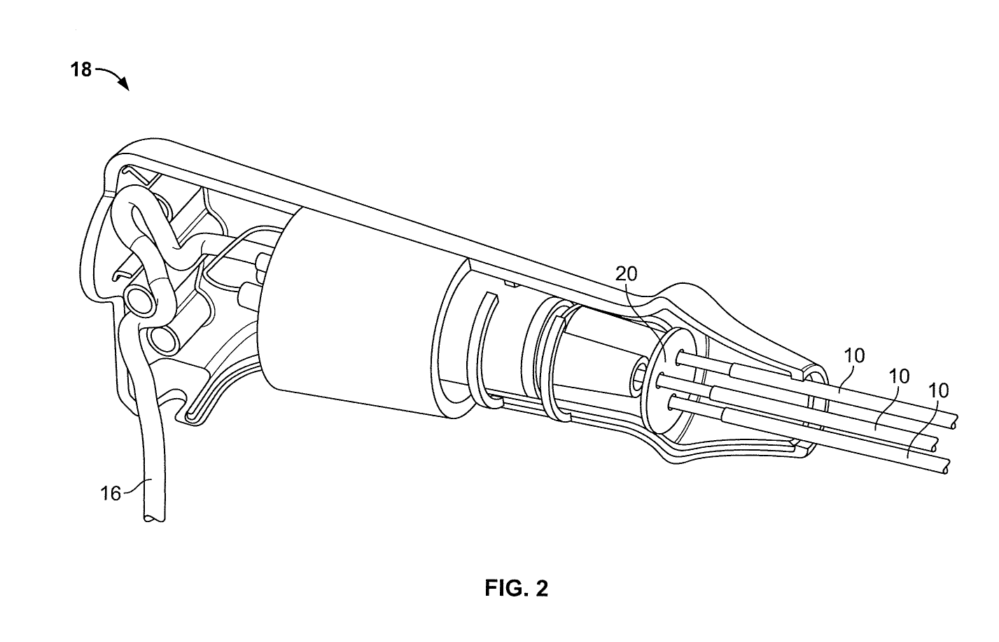

FIG. 2 illustrates the present invention to electrically short multiple needles together for use in a surgical device. The electro surgical device 18 houses multiple electro surgical needles 10 where only one needle 10 is electrically charged. The electrical shorting plate 20 is constructed of a conductive material and contacts each of the surgical needles 10, thereby conducting the electrical charge from one needle 10 to all of the needles 10 in the device 18.

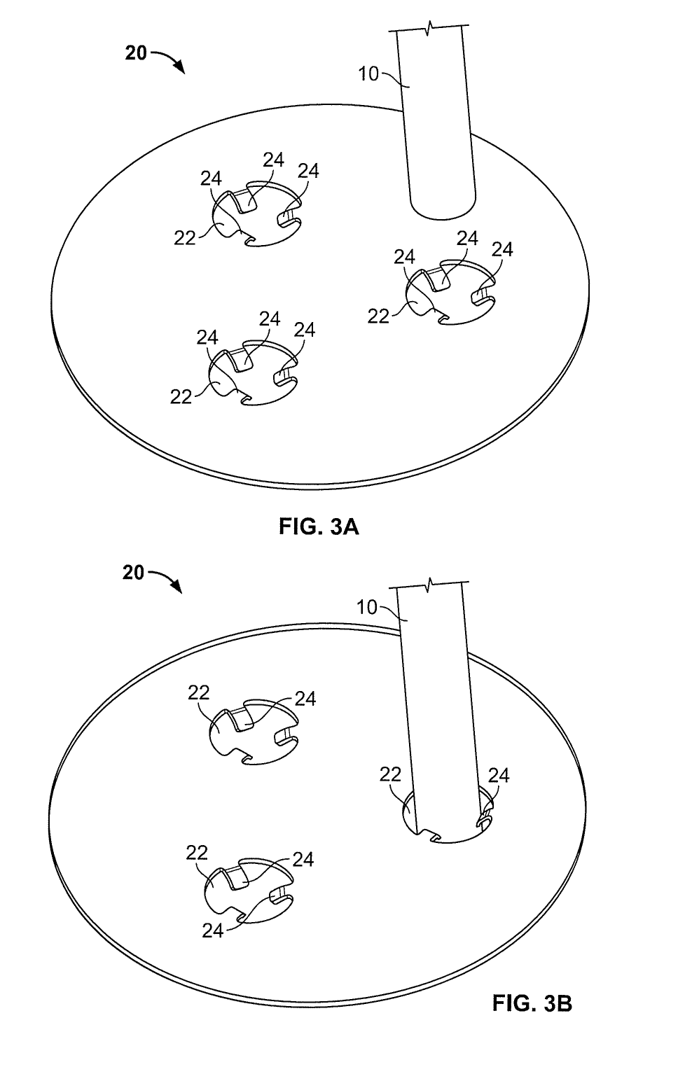

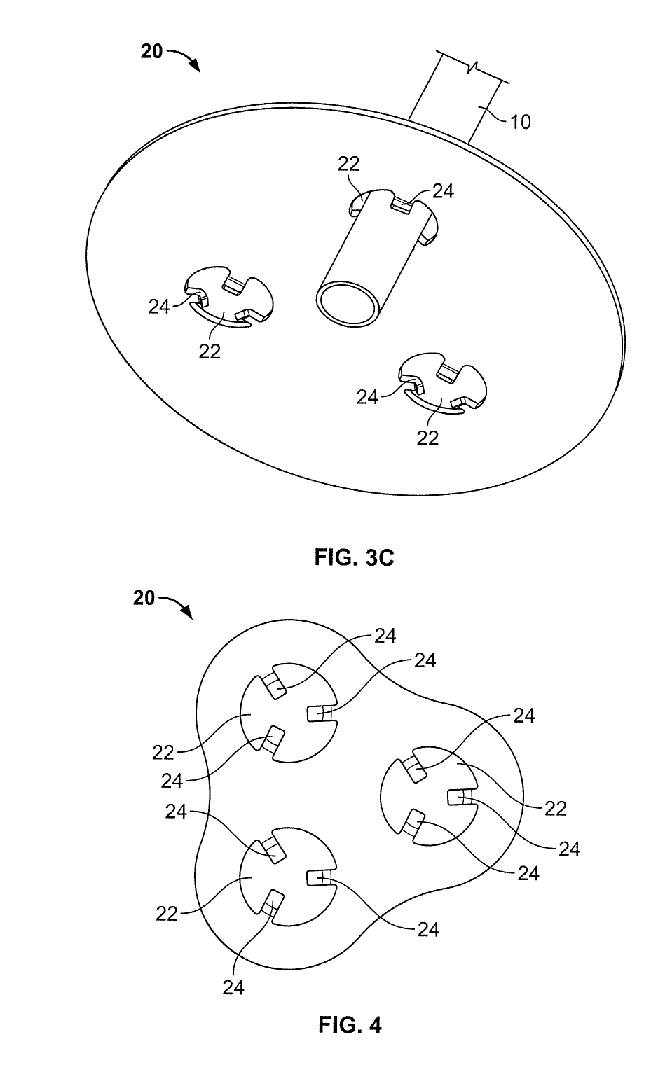

FIG. 3A illustrates one embodiment of the electrical shorting plate 20 in which the shape of the plate 20 is substantially circular. It is to be understood that while a substantially circular shorting plate 20 is shown, the plate 20 could be of any suitable shape. The plate 20 is shown having three apertures 22, however, it is to be appreciated that any multiple number of apertures 22 might be used with the shorting plate 20. FIG. 3A shows the shorting plate 20 prior to the insertion of RF energy needles 10 into apertures 22. Each aperture 22 is formed having multiple protrusions or fingers 24 that compressingly contact the needle 10 as well as providing a secure electrical connection to the shorting plate 20.

FIG. 3B shows the shorting plate 20 as a RF energy needle 10 is inserted into one of the apertures 22. As the needle 10 is inserted, the protrusions or fingers 24 contact the needle 10, providing an electrical connection. The fingers 24 are sufficiently resilient to elastically deflect in order to receive the needle 10 and provide a secure connection that maintains an elastically conductive connection as well as a mechanical retention on the needle 10. FIG. 3C shows the shorting plate 20 with a needle 10 fully inserted through one of the apertures 22. The fingers 24 are shown as contacting the needle 10 at a slightly greater angle than the fingers 24 in the other apertures 22. This is because the fingers 24 contacting the needle 10 have elastically deflected to receive the needle 10 and provide a secure connection and frictional hold on the needle 10 to prevent it from moving once inserted.

FIG. 4 illustrates another embodiment of the shorting plate 20 where the shape is substantially triangular. It should be understood that while a substantially triangular shape is shown, any suitable shape could be used. The shorting plate 20 is preferably stamped from a unitary piece of conductive metal. For example, a phosphorus-bronze material, one containing 98% copper can be used, as well as any copper based alloy. The plate 20 is preferably coated with tin, nickel, gold, silver or any other conductive material. Although the plate 20 can be any thickness suitable for use in the device 18, in one embodiment the plate is approximately 0.008 inches thick and finished with a chemical plating. Preferably, the plating is a tin material, but it is understood that the plating can be any suitable conductive and durable material. While the fingers 18 can be of any suitable size, in one embodiment the fingers may extend approximately 0.016 inches into the apertures 22, which are approximately 0.12 inches in diameter. However, it should be understood that the apertures 22 are not necessarily circular, and that fingers 18 and apertures can be any suitable length and size to comfortably accommodate an RF energy needle 10. In addition, the shorting plate 20 can have any suitable surface area, for example, the surface area may be approximately 0.215 square inches.

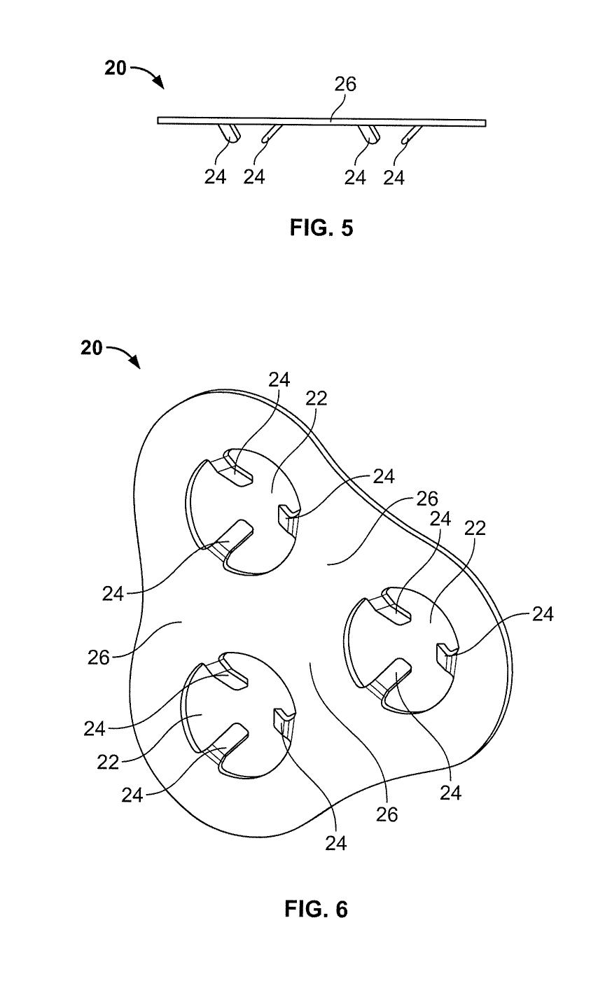

FIG. 5 illustrates a side view of the shorting plate 20 where the fingers 24 are visible as protruding below the body 26 of the plate 20. The fingers 18 are disposed at approximately a forty-five degree angle from the body 26 of the plate 20 before the needle 10 is inserted into the aperture 22. Once the needle 10 is inserted into the aperture 22, the fingers 24 resiliently deflect to accommodate the size of the needle 10 and are disposed at a larger angle than forty-five degrees, but no greater than approximately seventy-five degrees.

FIG. 6 illustrates a prospective view of the shorting plate 20. Once the plate 20 is stamped, the fingers 24 are preformed at an angle from the plate. This angle is preferably about forty-five degrees as measured from the body 26 of the plate. In addition, the plate may be stamped having as few as two apertures 22 for receiving needles 10 or any other number greater than two. Preferably, three apertures 22 are stamped into the plate 20, each aperture having three fingers 24. It is to be understood that alternate finger 24 profiles and apertures 22 profiles may permit the use of angles deviating both greater than or less than forty five degrees.

The shorting plate 20 can be configured for as low as two needles 10 and can accommodate any number of multiple needles 10 that is greater than two. In addition, the shorting plate 20 can accommodate a wide range of needles 10 varying in size and diameter because the fingers are configured to flexibly accept the needles 10 as they are inserted into the apertures 22 of the plate 20. In addition, the apertures 22 can be sized to accommodate any particularly sized needle 10 desired to fit in the plate 20. Once the needles 10 are inserted, no permanent connection, such as soldering, is necessary.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. An electrical shorting plate comprising:
an electrically conductive body;
a plurality of apertures formed in the electrical body, each aperture having at least one protrusion disposed at an angle from the body; and
wherein the body conducts an electrical charge to the plurality of protrusions, the apertures configured to receive an electrically conductive element, the at least one protrusion resiliently receiving the electrically conductive element and maintaining a secure electrical connection with the electrically conductive element.

2. The electrical shorting plate of claim 1 wherein the conductive body is substantially circular.

3. The electrical shorting plate of claim 1 wherein the conductive body is substantially triangular.

4. The electrical shorting plate of claim 1 wherein the plurality of apertures are substantially circular.

5. The electrical shorting plate of claim 1 wherein the plurality of apertures includes three apertures.

6. The electrical shorting plate of claim 1 wherein each aperture of the plurality of apertures contains three protrusions.

7. The electrical shorting plate of claim 1 wherein the at least one protrusion is disposed at an angle of about 45 degrees from the body.

8. The electrical shorting plate of claim 1 wherein the at least one protrusion extends at an angle downward from the conductive body.

9. The electrical shorting plate of claim 1 wherein the electrically conductive body is stamped formed.

10. An electrical surgical device comprising:
an electrical power source;
a plurality of electrically conductive elements where one of the electrically conductive elements is connected to the electrical power source and configured to receive an electrical charge;
an electrical shorting plate, further comprising;
an electrically conductive body;
a plurality of apertures formed in the electrical body, each aperture having at least one protrusion disposed at an angle from the body; and
wherein the body conducts an electrical charge to the at least one protrusion, the apertures configured to receive an electrically conductive element, the at least one protrusion resiliently receiving the electrically conductive element and maintaining a secure electrical connection with the electrically conductive element.

11. The electrical surgical device of claim 10 wherein the plurality of electrically conductive elements are surgical needles.

12. The electrical surgical device of claim 10 wherein the electrical shorting plate is substantially circular.

13. The electrical shorting plate of claim 10 wherein the plurality of apertures are substantially circular.

14. The electrical surgical device of claim 10 wherein the conductive body is substantially triangular.

15. The electrical surgical device of claim 10 wherein the plurality of apertures includes three apertures.

16. The electrical surgical device of claim 10 wherein each aperture of the plurality of apertures contains three protrusions.

17. The electrical surgical device of claim 10 wherein the at least one protrusion is disposed at an angle of about 45 degrees from the body.

18. The electrical shorting plate of claim 10 wherein the at least one protrusion extends at an angle downward from the conductive body.

19. The electrical shorting plate of claim 10 wherein the electrically conductive body is stamped formed.
